(19) United States
(12) Patent Application Publication
(10) Pub. No.: US 2024/0426760 A1
(43) Pub. Date: Dec. 26, 2024

(54) MOISTURE INDICATOR APPARATUS AND METHODS OF MAKING THEREOF

(71) Applicant: Sunrise Greenhouses LTD., Vineland (CA)

(72) Inventor: Rodney Bierhuizen, Vineland (CA)

(73) Assignee: Sunrise Greenhouses LTD., Vineland, ON (CA)

(21) Appl. No.: 18/702,698

(22) PCT Filed: Oct. 14, 2022
(86) PCT No.: PCT/CA2022/051523

§ 371 Date: Apr. 18, 2024

Related U.S. Application Data
(60) Provisional application No. 63/256,824, filed on Oct. 18, 2021.

(51) Int. Cl. G01N 21/78; A01G 9/02; A01G 27/04; C09J 5/06; C09J 7/35; G01N 33/24

(52) U.S. Cl. CPC G01N 21/78; C09J 5/06; C09J 7/35; G01N 33/241; A01G 9/02; A01G 27/04; C09J 2301/304

(57) ABSTRACT

This disclosure relates to a moisture indicator as well as methods of making the moisture indicator. The moisture indicator comprises a wick which comprises a lower portion insertable into a medium for absorbing water contained in the medium, an upper portion through which absorbed water evaporates and an indicator portion disposed between the lower and upper portions. The moisture indicator includes a hydrochromic material disposed on an indicator portion of the wick to provide a visual indication upon contact with moisture. The moisture indicator also includes a polymer layer sealingly covering the indicator portion and the hydrochromic material disposed thereon, thereby preventing water evaporation therethrough.

### CROSS-REFERENCE TO PREVIOUS APPLICATIONS

This application claims priority from U.S. provisional patent application No. 63/256,824 filed on Oct. 18, 2021, which is incorporated herein by reference in its entirety.

This disclosure generally relates to a moisture indicator. More specifically, this disclosure relates to a moisture indicator that comprises a hydrochromic material for providing a visual indication upon contact with moisture, and methods of making thereof.

### BACKGROUND

Moisture indicators for potted plants are known and available on the market. Some moisture indicators use batteries and measure capacitance and can be costly.

Moisture indicators using a wicking element and a chemical agent that changes color upon exposure to moisture are known. U.S. Pat. No. 8,997,682 B1 generally discloses a hydrochromic ink moisture indicator that reversibly changes between two different colors depending on whether the moisture indicator is in a wet state or in a dry state. The moisture indicator, which is preferably a stone-shaped body member, comprises a porous body member, preferably a porous ceramic, and is placed at the surface of the soil. Water passed through the porous material activates the moisture indicating material from a dry opaque state to a transparent clear state. In the transparent clear state, the moisture indicating material reveals the moisture indicating color.

Indicators inserted below the level of the soil may provide a more accurate indication of moisture in the soil and can help prevent overwatering. U.S. Pat. No. 3,951,098 generally discloses an indicator device for insertion into the potting soil of a conventional house plant, for monitoring the moisture content thereof. The indicator includes a generally flat plastic housing encapsulating an elongated wick extending from a lower point where it is exposed beneath the soil level to an upper point in an indicator chamber formed in the housing. The wick surrounds but does not touch a “signal element” comprising a layer of moisture-sensitive, color changeable substance (e.g., cobalt chloride), to carry moisture to the vicinity of the “signal element”. The substance changes color in response to predetermined gains (or losses) in moisture content.

U.S. Patent Application Publication No. 2016/0123867 A1 generally discloses a soil moisture indicator including a main body that is formed in a hollow shape from a material through which water does not pass and has a water absorption opening disposed near one end in a longitudinal direction, an evaporation opening disposed near the other end, and a display section provided near the other end and formed to visualize the hollow inside. A water absorption material e.g. cotton cloth, is filled on the inside of the main body and a water detection sheet is disposed to cover the water absorption material at a position of the display section inside the main body and has varying color tones between a water absorption state and a dry state.

There is a need for moisture indicators that are inexpensive and simple to manufacture and that can resist deterioration caused by salts contained in water, thus providing a longer lifespan. There is also a need for recyclable and/or biodegradable moisture indicators.

### SUMMARY

The following summary is intended to introduce the reader to various aspects of the applicant's teaching, but not to define or delimit any invention.

According to some aspects of this disclosure, there is provided a moisture indicator, comprising:
a wick comprising a lower portion insertable into a medium for absorbing water contained in the medium, an upper portion through which absorbed water evaporates and an indicator portion disposed between the lower and upper portions,
a hydrochromic material disposed on an indicator portion, the hydrochromic material providing a visual indication upon contact with moisture, and
a clear polymer layer sealingly covering the indicator portion and the hydrochromic material disposed thereon, the polymer layer preventing water evaporation therethrough.

In some examples, the wick is round, rectangular or square shaped.

In some examples, the polymer layer is an adhesive tape, shrink tubing or a hot melt adhesive.

In some examples, the adhesive tape is polyethylene tape.

In some examples, the hot melt adhesive is a rubber-based hot melt adhesive.

In some examples, the hydrochromic material is hydrochromic ink, hydrochromic paint or hydrochromic paper.

In some examples, the hydrochromic material changes color or opacity upon contact with moisture.

In some examples, the hydrochromic material is opaque when dry and becomes translucent upon contact with moisture.

In some examples, the wick is made of a natural material, optionally selected from reed, rattan, bamboo, wood, cotton, and mixtures thereof.

In some examples, the wick is made of a synthetic material, optionally selected from polyester, polyolefin, polyethylene, polyethylene terephthalate, nylon, polystyrene, and mixtures thereof.

In some examples, the wick is made of polyester. In some examples, the wick is made of polyethylene terephthalate.

In some examples, the wick is made of a combination of a synthetic material, optionally selected from polyester, polyolefin, polyethylene, nylon, polystyrene, polyethylene terephthalate, and mixtures thereof, and a natural material, optionally selected from reed, rattan, bamboo, wood, cotton, and mixtures thereof.

In some examples, the moisture indicator is recyclable and/or biodegradable.

In some examples, the hydrochromic material is applied as a symbol, a pattern, a word, or combinations thereof.

In some examples, the indicator further comprises a thermochromic material applied on the indicator portion.

In some examples, the indicator further comprises a photochromic material applied on the indicator portion.

In some examples, the wick has a length of about 5 cm to about 30 cm, about 10 cm to about 25 cm or about 15 cm to about 20 cm.

In some examples, the wick has a diameter of about 0.2 cm to about 2 cm, about 0.3 cm to about 1.8 cm, about 0.4 cm to about 1.6 cm, or about 0.5 cm to about 1.5 cm.

In some examples, the indicator portion has a length of about 0.5 cm to about 30 cm, about 1 cm to about 25 cm, about 1.5 cm to about 20 cm, about 2 cm to about 15 cm, about 2.5 cm to about 12 cm or about 2 cm to about 10 cm in length.

In some examples, an upper end of the indicator portion is about 0.5 cm to about 10 cm, about 1 cm to about 8 cm, about 1 cm to about 5 cm or about 1 cm to 3 cm from an upper end of the wick.

In some examples, a lower end of the indicator portion is about 0.5 cm to about 25 cm, 5 cm to about 22.5 cm, 10 cm to about 20 cm, or 15 cm to about 22 cm from a lower end of the wick.

In some examples, the medium is soil in a potted plant

In some examples, the medium is a liquid in a water reservoir.

According to some aspects of the present disclosure, there is provide a use of the moisture indicator herein described for measuring moisture in a potted plant.

According to some aspects of the present disclosure, there is provide a use of the moisture indicator herein described for measuring moisture in a water reservoir, optionally a reservoir of a self-watering pot.

In accordance with some aspects of this disclosure, a method of making a moisture indicator comprises:
a. providing a wick, the wick comprising a lower portion insertable into a medium for absorbing water contained in the medium, an upper portion through which absorbed water evaporates and an indicator portion disposed between the lower and upper portions,
b. providing a polymer layer,
c. applying to the polymer layer a hydrochromic material, the hydrochromic material providing a visual indication upon contact with moisture, and
d. applying the polymer layer over an indicator portion such that the hydrochromic material is transferred on the indicator portion and the polymer layer sealingly covers the indicator portion and the hydrochromic material transferred thereon.

In accordance with some aspects of this disclosure, a method of making a moisture indicators comprises:
a. providing a wick, the wick comprising a lower portion insertable into a medium for absorbing water contained in the medium, an upper portion through which absorbed water evaporates and an indicator portion disposed between the lower and upper portions,
b. applying to an indicator portion a hydrochromic material, the hydrochromic material providing a visual indication upon contact with moisture, and
c. applying a polymer layer over the indicator portion to sealingly cover the indicator portion and the hydrochromic material applied thereon.

In accordance with some aspects of this disclosure, a method of making a moisture indicators comprises:
a. providing a wick, the wick comprising a lower portion insertable into a medium for absorbing water contained in the medium, an upper portion through which absorbed water evaporates and an indicator portion disposed between the lower and upper portions,
b. applying a polymer layer over the indicator portion,
c. applying hydrochromic material at a surface between the polymer layer and the indicator portion, and
d. sealingly covering the polymer layer over the indicator portion and the hydrochromic material applied thereon.

In some examples, the wick is round, rectangular or share shaped.

In some examples, the polymer layer is shrink tubing.

In some examples, the polymer layer is an adhesive tape, optionally polyethylene tape or a hot melt adhesive, optionally a rubber-based hot melt adhesive.

In some examples, the polymer layer applied over the indicator portion is heated to a temperature of about 100° C. to about 200° C., about 120° C. to about 180° C., about 130° C. to about 170° C., 140° C. to about 160° C. or about 150° C., thereby sealingly covering the indicator portion and hydrochromic material thereon.

In some examples, the hydrochromic material is hydrochromic ink,

hydrochromic paint or hydrochromic paper.

In some examples, the hydrochromic ink is heated to a temperature of about 100° C. to about 200° C., 120° C. to about 180° C., 130° C. to about 170° C., or 140° C. to about 160° C. prior to applying to the polymer layer or the indicator portion.

In some examples, the hydrochromic ink is applied to the polymer layer or the indicator portion by stamping.

In some examples, the hydrochromic ink is applied to the polymer layer by screen printing.

In some examples, the hydrochromic material changes color or opacity upon contact with moisture.

In some examples, the hydrochromic material is opaque when dry and becomes translucent upon contact with moisture.

In some examples, the wick is made of a synthetic material, optionally selected from polyester, polyolefin, polyethylene, polyethylene terephthalate, nylon, polystyrene, and mixtures thereof.

In some examples, the wick is made of polyester. In some examples, the wick is made of polyethylene terephthalate.

In some examples, the wick is made of a combination of a synthetic material, optionally selected from polyester, polyolefin, polyethylene, polyethylene terephthalate, nylon, polystyrene, and mixtures thereof, and a natural material, optionally selected from reed, rattan, bamboo, wood, cotton, and mixtures thereof.

In some examples, the moisture indicator is recyclable and/or biodegradable.

In some examples, the hydrochromic material is applied as a symbol, a pattern, a word, or combinations thereof.

In some examples, the method further comprises applying a thermochromic material on the polymer layer or the indicator portion.

In some examples, the method further comprises applying a photochromic material the polymer layer or the indicator portion.

In some examples, the wick has a length of about 5 cm to about 30 cm, about 10 cm to about 25 cm or about 15 cm to about 20 cm.

In some examples, the wick has a diameter of about 0.2 cm to about 2 cm, about 0.3 cm to about 1.8 cm, about 0.4 cm to about 1.6 cm, or about 0.5 cm to about 1.5 cm.

In some examples, the indicator portion of has a length of about 0.5 cm to about 30 cm, about 1 cm to about 25 cm, about 1.5 cm to about 20 cm, about 2 cm to about 15 cm, about 2.5 cm to about 12 cm or about 2 cm to about 10 cm in length.

In some examples, an upper end of the indicator portion is about 0.5 cm to about 10 cm, about 1 cm to about 8 cm, about 1 cm to about 5 cm or about 1 cm to 3 cm from an upper end of the wick.

In some examples, a lower end of the indicator portion is about 0.5 cm to about 25 cm, 5 cm to about 22.5 cm, 10 cm to about 20 cm, or 15 cm to about 22 cm from a lower end of the wick.

According to some aspects of this disclosure, a kit comprises the moisture indicator herein disclosed and a plant pot.

In some examples, the kit further comprises a plant and a growing medium.

### BRIEF DESCRIPTION OF THE DRAWINGS

The drawings included herewith are for illustrating various examples of systems, apparatuses, and methods of the present specification and are not intended to limit the scope of what is taught in any way. In the drawings:

FIG. 1 is a perspective view of an example moisture indicator;

FIG. 2 is a perspective view of an example moisture indicator;

FIG. 3 is a perspective view of an example moisture indicator;

FIG. 4 is a perspective view of an example moisture indicator;

FIG. 5 is a perspective view of an example moisture indicator;

FIG. 6 is a partial close-up, cross-sectional view of the moisture indicator of FIG. 1;

FIG. 7 is a partial close-up, cross-sectional view of the moisture indicator of FIG. 5;

FIG. 8 is a flowchart of an exemplary method of making a moisture indicator in accordance with an embodiment;

FIG. 9 is a flowchart of an exemplary method of making a moisture indicator in accordance with another embodiment; and

FIG. 10 is a flowchart of an exemplary method of making a moisture indicator in accordance with another embodiment.

### DETAILED DESCRIPTION

Various apparatuses, systems, or methods will be described below to provide example embodiments of the claimed subject matter. No embodiment described below limits any claim and any claim may cover apparatuses, systems, or methods that differ from those described below. The claims are not limited to apparatuses, systems, or methods having all of the features of any one apparatus, system, or method described below or to features common to multiple or all of the apparatuses, systems and method described below. It is possible that an apparatus, system, or method described below is not an embodiment of any claim. Any subject matter disclosed in an apparatus, system, or method described below that is not claimed in this document may be the subject matter of another protective instrument, for example, a continuation patent application, and the applicants, inventors or owners do not intend to abandon, disclaim or dedicate to the public any subject matter by its disclosure in this document.

Moisture indicators can be used to provide a visual indication of the moisture content in a receptacle such as a potted plant or a water reservoir, such as a reservoir of a self-watering pot.

The inventor has developed an easy and inexpensive moisture indicator using a wick. A hydrochromic material is disposed on a wick portion and a polymer layer sealingly covers the wick portion and hydrochromic material disposed thereon. It has been found that the seal provided by the polymer layer prevents evaporation of water therethrough and prevents accumulation at the surface of the wick of salts contained in water and particularly in fertilizer solutions. The seal also prevents water sprayed near the wick to come into contact with the hydrochromic material. This may advantageously preserve the hydrochromic material and increase the overall lifespan of the moisture indicator. It has also been found that applying the polymer layer below the upper portion of the wick and leaving the upper portion exposed improves capillary action of the wick as well as evaporation of water through the upper portion.

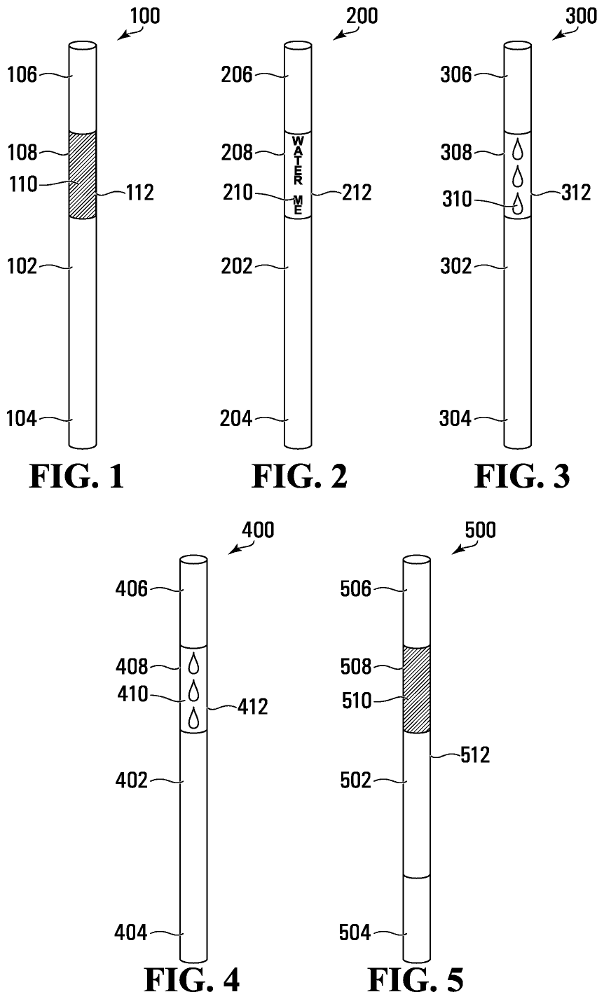

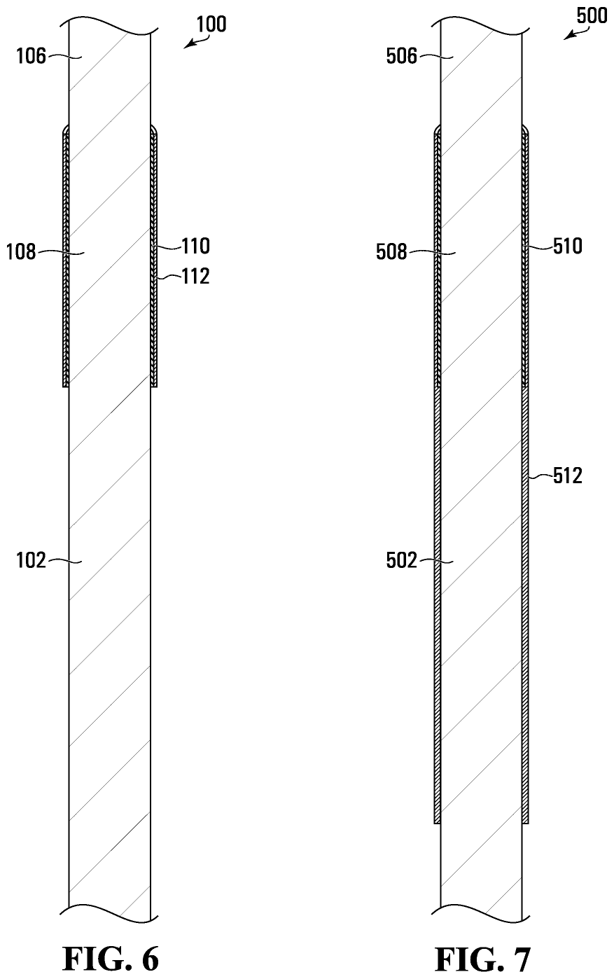

Now, referring to FIG. 1, an exemplary moisture indicator 100 is shown. In this embodiment, the moisture indicator 100 includes a wick 102. The wick 102 comprises a lower portion 104 insertable into a medium for absorbing water contained in the medium, an upper portion 106 through which absorbed water evaporates and an indicator portion 108 disposed between the lower 104 and upper 106 portions. A partial, close-up, cross-sectional view of the moisture indicator 100 is illustrated in FIG. 6.

It will be understood that the wick can be made of a variety of materials which can be natural, synthetic or a combination thereof. In some embodiments, the natural material(s) is/are reed, rattan, bamboo, wood, cotton, and mixtures thereof. In some embodiments, the synthetic material(s) is/are polyester, polyolefin, polyethylene, polyethylene terephthalate, nylon, polystyrene, and mixtures thereof. In one embodiment, the wick is made of polyester.

A person skilled in the art will appreciate that the suitability of the wicking material is determined by different factors such as rigidity, durability, ability to transport moisture and speed at which it wicks moisture. Other factors may include resistance to UV degradation and microbial resistance (e.g. bacteria, insects and mold).

Preferably, the wick is sufficiently rigid such that it can be inserted into a growing medium and retain its shape and sturdiness, even as it is wicking moisture. In embodiments where the wick is not sufficiently rigid to retain its shape, additional rigidity may be conferred by the polymer layer surrounding part of the wick. As further described below, the length of the polymer layer may vary. Where additional rigidity is desirable, the polymer layer may be longer. For example the polymer layer may extend along the length of the wick, while preferably leaving upper and lower portions exposed for ensuring sufficient wick capillary action as well as evaporation through the upper portion.

It will also be understood that various wick lengths and diameters may be contemplated. For example, where the moisture indicator is intended to be used to measure moisture in a smaller potted plant, a shorter and thinner wick may be desirable and where moisture indicator is for a bigger potted plant, a longer and thicker wick may be desirable. Depending on the plant type and root system, a smaller or larger wick may be desirable.

In some embodiments, the wick has a length of about 5 cm to about 30 cm, about 10 cm to about 25 cm or about 15 cm to about 20 cm.

In some embodiments, the wick has a diameter of about 0.2 cm to about 2 cm, about 0.3 cm to about 1.8 cm, about 0.4 cm to about 1.6 cm, or about 0.5 cm to about 1.5 cm.

Still referring to FIG. 1, the wick 102 comprises an indicator portion 18 on which is disposed a hydrochromic material 112.

As mentioned above, the hydrochromic material provides a visual indication upon contact with moisture. In use, when the moisture indicator is inserted into a medium (e.g. soil) and moisture in the medium is present, the moisture is absorbed from the lower portion 104 of the wick. Via capillary action, the moisture moves longitudinally along the length of the wick 102 towards the upper portion 106 of the wick through which it evaporates. When the moisture comes into contact with the indicator portion 108, the hydrochromic material 110 in turn becomes wet and changes color or opacity, thus providing the visual indication of moisture. When the wick is dry, the hydrochromic material reversibly changes.

In some embodiments, the hydrochromic material when dry is opaque, for example opaque white, and upon contact with moisture becomes clear. Depending on the type of hydrochromic material, it may be translucent or preferably transparent when wet. To readily detect the visual indication, the color of the indicator portion contrasts that of the hydrochromic material. For example, where the hydrochromic material is white when dry the wick or indicator portion is preferably a bright or dark color but is not white.

There are a number of ways in which the visual indication may be provided. Referring to FIGS. 1 and 6, the hydrochromic material 110 is disposed on the entire indicator portion 108 and extends around the entire circumference of the indicator portion 108 (not shown). For example, the hydrochromic material 110 may be sprayed onto the entire indicator portion 108, or a substantial part of the indicator portion 108, in a band.

It will be understood that the hydrochromic material may be disposed on only a part of the indicator portion. For example, the hydrochromic material may be applied on the indicator portion as symbol(s), pattern(s), word(s) or a combination thereof, as illustrated in FIGS. 2 to 4.

Now referring to FIG. 2, another exemplary moisture indicator is shown. Like elements are referred to using like reference numerals, incremented by 100. Unless otherwise stated below, all the teachings in relation to the moisture indicator 100, its features and/or components may apply to the moisture indicators 200, 300, 400 and 500 as well.

The moisture indicator 200 includes a wick 202. The wick 202 comprises a lower portion 204 insertable into a medium for absorbing water contained in the medium, an upper portion 206 through which absorbed water evaporates and an indicator portion 208 disposed between the lower 204 and upper 206 portions. A hydrochromic material 210 is disposed on an indicator portion 208. In this example, the hydrochromic material 210 is applied as words “WATER ME” which becomes apparent when the wick is dry. As used herein, the term “apparent” means that the hydrochromic material becomes more or less visible. The extent of the change in appearance may vary so long as it is detectable to the naked eye. For example, when the hydrochromic material become apparent (i.e. when the wick is dry) it may transition from being invisible (or transparent or translucent) to being visible or opaque. The degree of change in appearance may be lesser. For example the hydrochromic material may transition from invisible to slightly visible, from transparent to slightly opaque, or from slightly opaque to increased opacity.

With reference now to FIG. 3, the moisture indicator 300 includes a wick 302. The wick 302 comprises a lower portion 304 insertable into a medium for absorbing water contained in the medium, an upper portion 306 through which absorbed water evaporates and an indicator portion 308 disposed between the lower 304 and upper 306 portions. A hydrochromic material 310 is disposed on an indicator portion 308. In this example, the hydrochromic material 310 is applied as symbols, namely three water drops which become apparent when the wick is dry.

In yet another example, with reference to FIG. 4, the moisture indicator 400 includes a wick 402. The wick 402 comprises a lower portion 404 insertable into a medium for absorbing water contained in the medium, an upper portion 406 through which absorbed water evaporates and an indicator portion 408 disposed between the lower 404 and upper 406 portions. A hydrochromic material 410 is disposed on an indicator portion 408. In this example, the hydrochromic material 410 is applied as the outline and background of symbols, namely of three water drops which become apparent when the wick is dry. In this example, when the wick is dry, the drops are the color of the indicator portion 408 and the background is the color of the hydrochromic material 410, e.g. white.

In an embodiment, the hydrochromic material is hydrochromic ink, hydrochromic paint or hydrochromic paper. In an embodiment, the hydrochromic material is hydrochromic ink.

As mentioned above, it has been found that sealingly covering the indicator portion and the hydrochromic material disposed thereon with a polymer layer prevents evaporation of water therethrough and prevents accumulation at the surface of the indicator wick portion of salts contained in water and particularly in fertilizer solutions. As used herein, “sealingly” means that the polymer layer covers the indicator portion of the wick, on which is disposed the hydrochromic material, in such way that water or other liquids (e.g. electrolyte or fertilizer solutions) absorbed in the wick do not evaporate through the indicator portion nor do they accumulate between the indicator portion and the polymer layer. Similarly, as water does not evaporate through the indicator portion, salts (which may accumulate at other surfaces of the wick, for example at a top end of the wick) do not accumulate at a surface between the indicator portion of the wick and the polymer layer, thereby also extending the lifespan of the hydrochromic material and preventing damage thereto cause by the salts. The seal provided by the polymer layer also prevents water sprayed near the wick to come into contact with the hydrochromic material.

Any suitable polymer material known in the art may be used. The polymer is clear, preferably transparent, so that the change in appearance of the hydrochromic material may be easily observed. For example, the polymer layer is an adhesive tape, such as polyethylene tape, shrink tubing or a hot melt adhesive, such as a rubber-based hot melt adhesive. In some embodiments, the polymer layer is printed with symbol(s), pattern(s), word(s) or a combination thereof in a colour that is similar, or substantially identical, to the colour of the indicator portion 108. The indicator portion may also be covered with a hydrochromic material that is translucent, and preferably transparent, when wet, and opaque and of a different color to the indicator portion 108 when dry. Thus, when the hydrochromic material is wet, the symbol(s), pattern(s), word(s) or a combination thereof, printed on the polymer layer are substantially the same colour as their background (i.e., the colour of the indicator portion beneath the translucent, and preferably transparent, wet hydrochromic material), while when the hydrochromic material is dry, the symbol(s), pattern(s), word(s) or a combination thereof, printed on the polymer layer are of a different colour as their background (i.e., the colour of the dry hydrochromic material).

The polymer layer is preferably waterproof. Such material when applied around a wick portion prevents evaporation of water through the longitudinal surface of the wick portion. This also enhances capillary action, drawing water from the lower portion to the upper portion of the wick for evaporation therethrough. A waterproof polymer layer is in addition desirable as it does not provide a false reading of the moisture level in the medium if for example water is sprayed unintentionally to an exposed wick portion e.g. above soil level.

It will be understood that the wick can be of any shape. For example, without limitation, the wick can be round shaped (as exemplified in FIGS. 1 to 7), triangular shaped, square shaped, rectangular shaped or star shaped. In embodiments where the wick is round, egg-shaped or oval shaped, the indicator portion can consist of the entire circumferential surface of a cross sectional wick portion disposed between the lower and upper wick portions. Alternatively, the indicator portion can be a part of the circumferential surface. The polymer layer can for example cover the entire circumferential surface or a part thereof. Minimally, the polymer layer covers the indicator portion. In embodiments where the wick has flat surfaces, for example triangular shaped, square shaped, rectangular shaped (e.g. coffee stir stick or popsicle® stick shape) wicks, the indicator portion can consist of the entire outer surface of a cross-sectional wick portion disposed between the lower and upper wick portions. Alternatively, the indicator portion can for example be one or more surfaces of the cross-sectional wick portion. The polymer layer can for example cover the entire outer surface or a part thereof.

The polymer layer can also cover more than the indicator portion. In an embodiment, the polymer layer covers the entire wick with the exception of the upper and lower portions which remain exposed or uncovered.

Now, referring to FIGS. 5 and 7, the exemplary moisture indicator 500 includes a wick 502. The wick 502 comprises a lower portion 504 insertable into a medium for absorbing water contained in the medium, an upper portion 506 through which absorbed water evaporates and an indicator portion 508 disposed between the lower 504 and upper 506 portions. A hydrochromic material 510 is disposed on the indicator portion 508. As can be seen in particular in FIG. 7 which is a partial, close-up, cross-sectional view of the moisture indicator 500, the polymer layer 512 is longer than the indicator portion 508 and defined by the lower 504 and upper 506 portions.

The indicator portions of FIGS. 1 to 4 have a length of about 5 cm however it will be understood that other lengths are contemplated. In some embodiments, the indicator portion has a length of about 0.5 cm to about 30 cm, about 1 cm to about 25 cm, about 1.5 cm to about 20 cm, about 2 cm to about 15 cm, about 2.5 cm to about 12 cm or about 2 cm to about 10 cm.

In some embodiments, the polymer layer has the same length as the indicator portion. For example, referring back to FIGS. 1 to 4 and 6, the polymer layer 112, 212, 312 and 414, respectively, has the same length as the indicator portion 108, 208, 308 and 408.

In other embodiments, the polymer layer is longer than the indicator portion. As shown in FIGS. 5 and 7, the polymer layer 512 is longer than the indicator portion 508.

For example, the polymer layer has a length of about 0.5 cm to about 30 cm, about 1 cm to about 25 cm, about 1.5 cm to about 20 cm, about 2 cm to about 15 cm, about 2.5 cm to about 12 cm or about 2 cm to about 10 cm.

Preferably, the upper portion of the wick is at least 0.5 cm in length. In some embodiments, an upper end of the indicator portion is about 0.5 cm to about 10 cm, about 1 cm to about 8 cm, about 1 cm to about 5 cm or about 1 cm to 3 cm from the upper end of the wick. Preferably, the lower portion of the wick is at least 0.5 cm in length. In some embodiments, a lower end of the indicator portion is about 0.5 cm to about 25 cm, 5 cm to about 22.5 cm, 10 cm to about 20 cm, or 15 cm to about 22 cm from the lower end of the wick.

In one embodiment, the polymer layer extends the lower and upper ends of the indicator portion by at least 0.1 cm, 0.2 cm, 0.3 cm, 0.4 cm, 0.5 cm, 1 cm or 2 cm to ensure adequate seal of the hydrochromic material.

In addition to the hydrochromic material, other materials may be applied on the indicator portion. Other materials that may be used in addition to the hydrochromic material include a photochromic material. In such embodiment, the indicator portion containing the photochromic material is activated by UV rays from the sun. Depending on the plant species and whether it thrives under full sun, partial sun or shade, this may prompt the user to modify sun exposure accordingly.

In some embodiments, the indicator portion may in addition comprise a thermochromic (or thermochromatic) material. For example, the thermochromic ink may become translucent when heat is applied, for example when the temperature exceeds 30° C. In use, the indicator portion provides a cue that the environment, e.g. the soil, is too hot and prompts the user to move the plant elsewhere, for example a shaded area, or cooler room.

In an embodiment, the moisture indicator is biodegradable and/or recyclable. As mentioned herein, the wick can be made of natural fibers which are biodegradable and/or recyclable. The polymer layer and hydrochromic material may also be biodegradable and/or recyclable along with photochromic and thermochromic materials.

In use, the moisture indicator herein described is inserted into a medium by way of its lower portion. For example, the medium is soil and the soil is contained in a potted plant. The moisture indicator can be inserted into the medium for example through a top opening of the pot or planter. The moisture indicator can be inserted vertically, with the upper end positioned upwardly and the lower end downwardly, but it can also be in inserted in any direction, for example sideways and upside-down (e.g. into a side or bottom aperture of the pot or planter) and would still work through capillarity and evaporation. In another example, the moisture indicator is inserted into a reservoir, optionally a reservoir of a self-watering pot. As will be understood, the depth of insertion of the moisture indicator in the medium will depend on the length of the container, the length of the moisture indicator as well as the type of plant. The indicator portion preferably remains exposed for ease of detection of visual indication of moisture.

It will be understood that depending on the plant water requirements, the moisture indicator can be placed closer or farther away from the center of the pot. For example, for a potted plant with high moisture needs, the moisture indicator may be placed closer to the edge of the pot, in other words away from the center. For example, for a cactus or succulent, it may be desirable to place the moisture indicator closer to the middle of the pot.

It will also be understood that the presently disclosed moisture indicator may be used to monitor the water level of a reservoir of a self-watering pot.

In some embodiments, the polymer layer covers more than the indicator portion, leaving only the lower and upper portions exposed. In such case a portion of the polymer layer may be inserted in the medium. It will be understood that the indicator portion must be at least partially exposed in order to easily detect the visual indication of moisture.

Methods of making a moisture indicator are also disclosed herein.

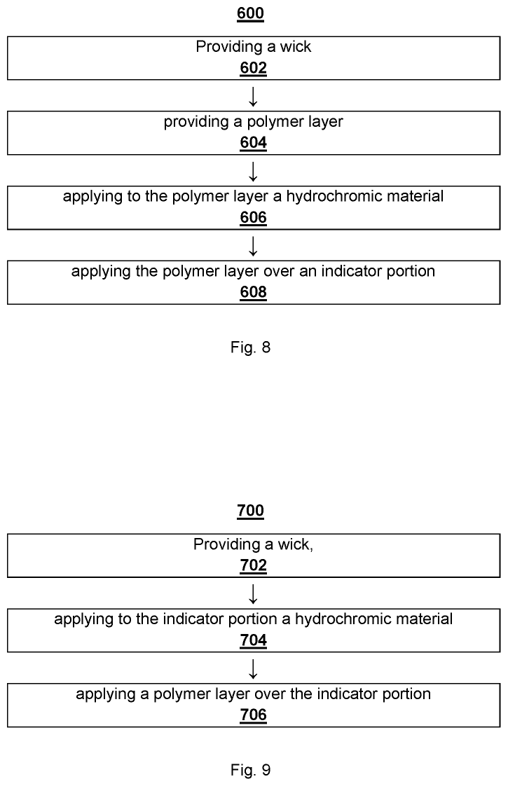

FIG. 8 illustrates a flow chart 600 of an exemplary method of making a moisture indicator. The method comprises at step 602 providing a wick, the wick comprising a lower portion insertable into a medium for absorbing water contained in the medium, an upper portion through which absorbed water evaporates and an indicator portion disposed between the lower and upper portions. At step 604, a polymer layer is provided and at step 606, a hydrochromic material is applied to the polymer layer. At step 608, the polymer layer is then applied over an indicator portion of the wick, transferring the hydrochromic material on the indicator portion and the polymer layer and sealingly covering the indicator portion and the hydrochromic material transferred thereon.

FIG. 9 illustrates a flow chart 700 of another exemplary method of making a moisture indicator. The method comprises at step 702 providing a wick, the wick comprising a lower portion insertable into a medium for absorbing water contained in the medium, an upper portion through which absorbed water evaporates and an indicator portion disposed between the lower and upper portions. The method further comprises at step 704 applying to the indicator portion a hydrochromic material, the hydrochromic material providing a visual indication upon contact with moisture, and at step 706 applying a polymer layer over the indicator portion to sealingly cover the indicator portion and the hydrochromic material applied thereon.

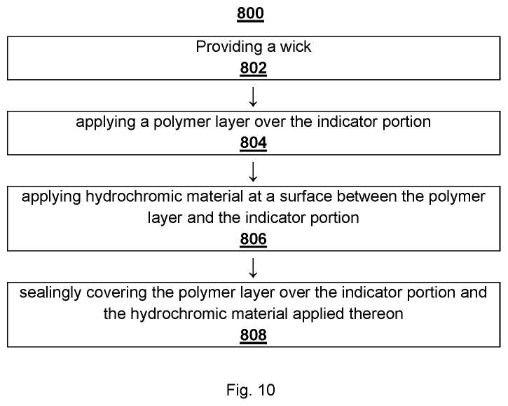

FIG. 10 illustrates a flow chart 800 of another exemplary method of making a moisture indicator. The method comprises at step 802 providing a wick, the wick comprising a lower portion insertable into a medium for absorbing water contained in the medium, an upper portion through which absorbed water evaporates and an indicator portion disposed between the lower and upper portions. The method further comprises at step 804 applying a polymer layer over the indicator portion, at step 806 applying hydrochromic material at a surface between the polymer layer and the indicator portion, and at step 808 sealingly covering the polymer layer over the indicator portion and the hydrochromic material applied thereon.

In some embodiments, the polymer layer applied over the indicator portion of the wick body is heated to a temperature of about 100° C. to about 200° C., about 120° C. to about 180° C., about 130° C. to about 170° C., 140° C. to about 160° C. or about 150° C., thereby sealingly covering the indicator portion of the wick body and hydrochromic material thereon.

In some embodiments, the hydrochromic ink is heated to a temperature of about 100° C. to about 200° C., 120° C. to about 180° C., 130° C. to about 170° C., or 140° C. to about 160° C. prior to applying to the polymer layer or the indicator portion of the wick body.

The following non-limiting examples are illustrative of the present disclosure:

### EXAMPLES

### Example 1

Hydrochromic ink was obtained from SFXC (https://www.sfxc.co.uk/). The ink when wet turns from white to translucent. Hydrochromic ink bands of about 2 inches in length were applied at about one inch from the top end of wicks. It was hypothesized that the one inch untreated top is where the salt contained in the fertilizer would accumulate. Further, a plastic layer was applied around the ink to prevent evaporation. It was found that the plastic layer prevents the water from evaporating where the ink is applied so that the salt does not damage the ink.

### Example 2

Testing was conducted with shrink tubing covering a portion of the wick. A syringe was used to coat the inside of the tube with hydrochromic ink. The shrink tubing squeezes the wick to ensure a good contact with the ink and transparency when the ink is wet. The prepared wicks were inserted into the soil of potted plants (Campanula plugs) which received fertilizer (high EC). After 40 weeks of testing, the wicks were still functioning.

### Example 3

The hydrochromic ink was applied to plastic tape using a silk screen application. The plastic tape was then wrapped around the wick. This provided desirable results.

### Example 4

Wicks were wrapped with Uline clear tape previously screen printed with hydrochromic ink. the tape covered the wicks expect for 1 cm exposed at the top end and 1 cm exposed at the bottom end. A heat gun was used to dry and shrink the tape. This method also provided desirable results.

### Example 5

Wicks were covered with an 8 mm by 2 inch strip of hydrochromic ink, 1 cm down from the top end of the wick. The wicks were then wrapped with clear tape on their entire length except 1 cm from the top end and 2 cm from the bottom end. It was found that when the ink was heated prior to applying to the wick the ink layer stayed whiter.

### Example 6

Testing was conducted to apply dry hydrochromic ink to the wicks. A screen

printed text was designed so that words would appear when the wick is dry. In this example the words WATER ME were screen printed on clear Uline tape. Heating the wick to melt the tape allowed the tape staying wrapped around the wick, providing desirable results.

### Example 7

Testing was conducted using recyclable materials. A heavy coat rubber-based hot melt adhesive was used. The hydrochromic ink was applied to the adhesive side of the tape and the adhesive was then heat treated to 150° C. before application to a polyethylene terephthalate wick (1 cm below the top of the wick). The wicks were tested for 27 weeks with no visible salt or discoloration damage observed. It is believed that the adhesive prevents salts from infiltrating between the label (adhesive) and the ink.

While the above description provides examples of one or more apparatuses, articles, systems or methods, it will be appreciated that other apparatuses, articles, systems or methods may be within the scope of the accompanying claims, e.g., any other moisture indicator that includes any permutations or combinations of features and/or components disclosed herein.

1. A moisture indicator, comprising:
a wick comprising a lower portion insertable into a medium for absorbing water contained in the medium, an upper portion through which absorbed water evaporates and an indicator portion disposed between the lower and upper portions,
a hydrochromic material disposed on the indicator portion, the hydrochromic material providing a visual indication upon contact with moisture, and
a clear polymer layer sealingly covering the indicator portion and the hydrochromic material disposed thereon, the polymer layer preventing water evaporation therethrough.

2. The indicator of claim 1, wherein the wick is round, square or rectangular shaped.

3. The indicator of claim 1, wherein the polymer layer is an adhesive tape, shrink tubing or a hot melt adhesive.

4. The indicator of claim 3, wherein the adhesive tape is polyethylene tape.

5. The indicator of claim 3, wherein the hot melt adhesive is a rubber-based hot melt adhesive.

6. The indicator of claim 1, wherein the hydrochromic material is hydrochromic ink, hydrochromic paint or hydrochromic paper.

7. The indicator of claim 1, wherein the hydrochromic material changes color or opacity upon contact with moisture.

8. (canceled)

9. The indicator of claim 1, wherein the wick is made of a natural material, optionally selected from reed, rattan, bamboo, wood, cotton, and mixtures thereof.

10. The indicator of claim 1, wherein the wick is made of a synthetic material, optionally selected from polyester, polyolefin, polyethylene, polyethylene terephthalate, nylon, polystyrene, and mixtures thereof.

11.-16. (canceled)

17. The indicator of claim 1, wherein the wick has a length of about 5 cm to about 30 cm, about 10 cm to about 25 cm or about 15cm to about 20 cm.

18. The indicator of claim 1, wherein the wick has a diameter of about 0.2 cm to about 2 cm, about 0.3 cm to about 1.8 cm, about 0.4 cm to about 1.6 cm, or about 0.5 cm to about 1.5 cm.

19. The indicator of claim 1, wherein the indicator portion has a length of about 0.5 cm to about 30 cm, about 1 cm to about 25 cm, about 1.5 cm to about 20 cm, about 2 cm to about 15 cm, about 2.5 cm to about 12 cm or about 2 cm to about 10 cm in length.

20.-25. (canceled)

26. A method of making a moisture indicator, comprising:
a. providing a wick, the wick comprising a lower portion insertable into a medium for absorbing water contained in the medium, an upper portion through which absorbed water evaporates and an indicator portion disposed between the lower and upper portions,
b. providing a polymer layer,
c. applying to the polymer layer a hydrochromic material, the hydrochromic material providing a visual indication upon contact with moisture, and
d. applying the polymer layer over an indicator portion such that the hydrochromic material is transferred on the indicator portion and the polymer layer sealingly covers the indicator portion and the hydrochromic material transferred thereon.

27.-28. (canceled)

29. The method of claim 26, wherein the wick is round, rectangular or square.

30. The method of claim 26, wherein the polymer layer is shrink-tubing.

31. The method of claim 26, wherein the polymer layer is an adhesive tape, optionally polyethylene tape or a hot melt adhesive, optionally a rubber-based hot melt adhesive.

32. The method of claim 26, wherein the polymer layer applied over the indicator portion is heated to a temperature of about 100° C. to about 200° C., about 120° C. to about 180° C., about 130° C. to about 170° C., 140° C. to about 160° C. or about 150° C., thereby sealingly covering the indicator portion and hydrochromic material thereon.

33. The method of claim 26, wherein the hydrochromic material is hydrochromic ink, hydrochromic paint or hydrochromic paper.

34. The method of claim 33, wherein the hydrochromic ink is heated to a temperature of about 100° C. to about 200° C., 120° C. to about 180° C., 130° C. to about 170° C., or 140° C. to about 160° C. prior to applying to the polymer layer or the indicator portion.

35. The method of claim 33, wherein the hydrochromic ink is applied to the polymer layer or the indicator portion by stamping or by screen printing.

36.-49. (canceled)

50. A kit comprising the moisture indicator of claim 1 and a plant pot.

51. (canceled)
